(19) United States
(12) Patent Application Publication
(10) Pub. No.: US 2019/0099515 A1
(43) Pub. Date: Apr. 4, 2019

(54) IMPLANTABLE FUSION DEVICES COMPRISING BIOACTIVE GLASS

(71) Applicant: PROSIDYAN, INC., New Providence, NJ (US)

(72) Inventors: Charanpreet S. BAGGA, Basking Ridge, NJ (US); Shrikar P. Bondre, East Windsor, NJ (US); Steven B. Jung, Rolla, MO (US); Hyun W. Bae, Los Angeles, CA (US)

(21) Appl. No.: 16/151,774

(22) Filed: Oct. 4, 2018

Related U.S. Application Data
(60) Provisional application No. 62/568,086, filed on Oct. 4, 2017.

(51) Int. Cl. A61L 27/10; A61F 2/44; A61L 27/54; A61L 27/58

(52) U.S. Cl. CPC A61L 27/10; A61F 2/446; A61F 2002/30224; A61L 27/58; A61L 27/54

(57) ABSTRACT

Implantable devices that comprise an improved bone graft material, such as for example, bioactive glass, are disclosed. Additionally, implantable devices that work in conjunction with an improved bone graft material and act as a composite implantable device, for the improved treatment of bone, are also disclosed. These devices are bioactive, and are engineered to provide enhanced cellular activity to promote bone fusion or regrowth.

### CROSS-REFERENCE TO RELATED APPLICATIONS

This application claims benefit of U.S. Provisional No. 62/568,086 filed Oct. 4, 2017, which is herein incorporated by reference in its entirety.

### TECHNICAL FIELD

The present disclosure relates generally to implantable devices for treating bone. More particularly, the disclosure relates to implantable fusion devices comprising bioactive glass and/or containing a bone graft component of bioactive glass, and methods of using such devices for bone tissue regeneration and/or repair.

### BACKGROUND

A common surgical treatment to repair or replace damaged bone in a patient's body is to implant a device at the location of the damage that can facilitate bone fusion or bone regrowth. For example, specific to the spine, one method of repair is to remove the damaged vertebra (in whole or in part) and/or the damaged disc (in whole or in part) and replace it with an implant or prosthesis. In some cases, it is necessary to stabilize a weakened or damaged spinal region by reducing or inhibiting mobility in the area to avoid further progression of the damage and/or to reduce or alleviate pain caused by the damage or injury. In other cases, it is desirable to join together the damaged vertebrae and/or induce healing of the vertebrae. Accordingly, an implant or prosthesis may be configured to facilitate fusion between two adjacent vertebrae. The implant or prosthesis may be placed without attachment means or fastened in position between adjacent structural body parts (e.g., adjacent vertebral bodies).

Most bone fusion implants are configured mainly to provide a rigid structural framework to support new bone growth at the area of weakened bone. However these implants themselves do not necessarily promote new growth in and of themselves. Rather, these implants immobilize and/or stabilize the damaged area to reduce further damage. The implants must work in conjunction with an additional bone growth enhancing component to aid in the bone regrowth and/or repair process. For instance, the implants may be coated with a biological agent that promotes bone growth. Quite often these implants will serve as cages, and include a compartment to hold bone graft material to facilitate fusion.

The role of bone graft materials in clinical applications to aid the healing of bone has been well documented over the years. Most bone graft materials that are currently available, however, have failed to deliver the anticipated results necessary to make these materials a routine therapeutic application in reconstructive surgery. Improved bone graft materials for forming bone tissue implants that can produce reliable and consistent results are therefore still needed and desired.

In recent years intensive studies have been made on bone graft materials in the hopes of identifying the key features necessary to produce an ideal bone graft implant, as well as to proffer a theory of the mechanism of action that results in successful bone tissue growth. At least one recent study has suggested that a successful bone tissue scaffold should consider the physicochemical properties, morphology and degradation kinetics of the bone being treated. (“Bone tissue engineering: from bench to bedside”, Woodruff et al., Materials Today, 15(10): 430-435 (2012)). According to the study, porosity is necessary to allow vascularization, and the desired scaffold should have a porous interconnected pore network with surface properties that are optimized for cell attachment, migration, proliferation and differentiation. At the same time, the scaffold should be biocompatible and allow flow transport of nutrients and metabolic waste. Just as important is the scaffold's ability to provide a controllable rate of biodegradation to compliment cell and/or tissue growth and maturation. Finally, the ability to model and/or customize the external size and shape of the scaffold is to allow a customized fit for the individual patient is of equal importance.

Woodruff, et. al. also suggested that the rate of degradation of the scaffold must be compatible with the rate of bone tissue formation, remodeling and maturation. Recent studies have demonstrated that initial bone tissue ingrowth does not equate to tissue maturation and remodeling. According to the study, most of the currently available bone graft materials are formulated to degrade as soon as new tissue emerges, and at a faster rate than the new bone tissue is able to mature, resulting in less than desirable clinical outcomes.

Other researchers have emphasized different aspects as the core features of an ideal bone graft material. For example, many believe that the material's ability to provide adequate structural support or mechanical integrity for new cellular activity is the main factor to achieving clinical success, while others emphasize the role of porosity as the key feature. The roles of porosity, pore size and pore size distribution in promoting revascularization, healing, and remodeling of bone have long been recognized as important contributing factors for successful bone grafting implants. Many studies have suggested an ideal range of porosities and pore size distributions for achieving bone graft success. However, as clinical results have shown, a biocompatible bone graft having the correct structure and mechanical integrity for new bone growth or having the requisite porosities and pore distributions alone does not guarantee a good clinical outcome. What is clear from this collective body of research is that the ideal bone graft material should possess a combination of structural and functional features that act in synergy to allow the bone graft material to support the biological activity and an effective mechanism of action as time progresses.

Currently available bone graft materials fall short of meeting these requirements. That is, many bone graft materials tend to suffer from one or more of the problems previously mentioned, while others may have different, negatively associated complications or shortcomings. One example is autograft implants. Autograft implants have acceptable physical and biological properties and exhibit the appropriate mechanical structure and integrity for bone growth. However, the use of autogenous bone requires the patient to undergo multiple or extended surgeries, consequently increasing the time the patient is under anesthesia, and leading to considerable pain, increased risk of infection and other complications, and morbidity at the donor site.

When it comes to synthetic bone graft substitutes, the most rapidly expanding category consists of products based on calcium sulfate, hydroxyapatite and tricalcium phosphate. Whether in the form of injectable cements, blocks or morsels, these materials have a proven track record of being effective, safe bone graft substitutes for selected clinical applications. Recently, new materials such as bioactive glass (“BAG”) have become an increasingly viable alternative or supplement to natural bone-derived graft materials. In comparison to autograft implants, these new synthetic implants have the advantage of avoiding painful and inherently risky harvesting procedures on patients. Also, the use of these synthetic, non-bone derived materials can reduce the risk of disease transmission. Like autograft and allograft implants, these new artificial implants can serve as osteoconductive scaffolds that promote bone regrowth. Preferably, the graft implant is resorbable and is eventually replaced with new bone tissue.

Many artificial bone grafts available today comprise materials that have properties similar to natural bone, such as implants containing calcium phosphates. Exemplary calcium phosphate implants contain type-B carbonated hydroxyapatite whose composition in general may be described as (Ca₅(PO₄)₃ₓ(CO₃)ₓ(OH)). Calcium phosphate ceramics have been fabricated and implanted in mammals in various forms including, but not limited to, shaped bodies and cements. Different stoichiometric implants, such as hydroxyapatite (HA), tricalcium phosphate (TCP), tetracalcium phosphate (TTCP), and other calcium phosphate (CaP) salts and minerals have all been employed in attempts to match the adaptability, biocompatibility, structure, and strength of natural bone. Although calcium phosphate based materials are widely accepted, they lack the ease of handling, flexibility and capacity to serve as a liquid carrier/storage media necessary to be used in a wide array of clinical applications. Calcium phosphate materials are inherently rigid, and to facilitate handling are generally provided as part of an admixture with a carrier material; such admixtures typically have an active calcium phosphate ingredient to carrier volume ratio of about 50:50, and may have a ratio as low as 10:90.

As previously mentioned, the roles of porosity, pore size and pore size distribution in promoting revascularization, healing, and remodeling of bone have been recognized as important contributing factors for successful bone grafting. Yet currently available bone graft materials still lack the requisite chemical and physical properties necessary for an ideal graft implant. For instance, currently available graft materials tend to resorb too quickly (e.g., within a few weeks), while some take too long (e.g., over years) to resorb due to the implant's chemical composition and structure. For example, certain implants made from hydroxyapatite tend to take too long to resorb, while implants made from calcium sulfate or β-TCP tend to resorb too quickly. Further, if the porosity of the implant is too high (e.g., around 90%), there may not be enough base material left after resorption has taken place to support osteoconduction. Conversely, if the porosity of the implant is too low (e.g., 10%,) then too much material must be resorbed, leading to longer resorption rates. In addition, the excess material means there may not be enough room left in the residual graft implant for cell infiltration. Other times, the graft implants may be too soft, such that any kind of physical pressure exerted on them during clinical usage causes them to lose the fluids retained by them.

Thus, in order to provide a better clinical solution for the repair and/or replacement of bone, improved implantable devices as well as improved bone graft materials are needed. It would be desirable to therefore provide an implantable device that comprises an improved bone graft material, such as for example, bioactive glass, or works in conjunction with an improved bone graft material, for even better clinical results. Embodiments of the present disclosure address these and other needs.

### SUMMARY

The present disclosure provides various implantable devices that comprise an improved bone graft material, such as for example, bioactive glass, or implantable devices that work in conjunction with an improved bone graft material and act as a composite implantable device, for the improved treatment of bone. These devices are bioactive, and are engineered to provide enhanced cellular activity to promote bone fusion or regrowth.

According to one aspect, an implantable device is provided. The implantable device can comprise a plurality of compressed bioactive glass fibers, the device having a shape and geometry configured for insertion between adjacent bone segments to facilitate bone fusion. In some embodiments, the device may further comprise a plurality of bioactive glass particulates. The bioactive glass fibers may be randomly oriented, or may be aligned with respect to one another. In order to provide a load-bearing device, the fibers can be sintered together. In one clinical application, the adjacent bone segments are vertebral bodies. If desired, the device may further be porous.

According to another aspect, a composite implantable device is provided. The composite implantable device can comprise a fusion cage component, and a bone graft component, the bone graft component comprising a plurality of bioactive glass fibers. The device may have a shape and geometry configured for insertion between adjacent bone segments to facilitate bone fusion. In some embodiments, the device may further comprise a plurality of bioactive glass particulates. The bioactive glass fibers may be randomly oriented, or may be aligned with respect to one another. In order to provide a load-bearing device, the fibers can be sintered together. In one clinical application, the adjacent bone segments are vertebral bodies. If desired, the device may further be porous. The fusion cage component may comprise the same or a different material than the bone graft component. For example, in one embodiment, the fusion cage component can comprise a metal or metal-alloy material.

In one exemplary embodiment, an implantable device is provided. The implantable device may comprise a main body comprising a plurality of compressed bioactive glass fibers and at least one bundle of compressed bioactive glass fibers within the main body, the main body and the at least one bundle having different fiber densities and porosities. The device may have a shape and geometry configured for insertion between adjacent bone segments to facilitate bone fusion. The main body and/or bundle of fibers may also further comprise a plurality of bioactive glass particulates.

In some embodiments, the bioactive glass fibers of the main body or at least one bundle may be randomly oriented. In other embodiments, the bioactive glass fibers of the main body or at least one bundle are aligned with respect to one another.

In some embodiments, the bioactive glass fibers of the main body or at least one bundle are sintered together. The device may comprise a plurality of bundles of compressed bioactive glass fibers within the main body. The plurality of bundles of compressed bioactive glass fibers may be equidistantly spaced apart from one another within the main body. The device may be shaped as a cylinder. The device may be porous, or bioresorbable. The rate of resorption of the main body may be different than the rate of resorption of the at least one bundle. The device may be configured to be load-bearing, and be configured for placement between vertebral bodies in the intervertebral space.

In some embodiments, the device may include a coating over the main body. The coating may be heat wrapped over the main body. Additionally, the implantable device may include a biological agent, such as a growth factor, synthetic factor, recombinant factor, allogenic factor, a stem cell, demineralized bone matrix (DBM), or cell signaling agent.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings and photographs, which are incorporated in and constitute a part of this specification, illustrate several embodiments of the disclosure and together with the description, serve to explain the principles of the disclosure.

FIG. 1 shows a bone graft component of a composite implantable device comprising bioactive glass fibers.

FIG. 2A shows a bone graft component of a composite implantable device comprising a plurality of bundles of uniformly aligned bioactive glass fibers.

FIG. 2B shows a bone graft component of a composite implantable device comprising plurality of bundles of randomly aligned bioactive glass fibers.

FIG. 3 shows a composite implantable device comprising a cage component and a bone graft component.

FIG. 4 shows a composite implantable device comprising a multi-part cage component and bone graft component.

FIG. 5 shows a cross-sectional view of a composite implantable device comprising a cage component and different bone graft components associated therewith.

FIG. 6 shows another composite implantable device comprising a cage component and bone graft component contained therein.

FIG. 7 shows still another composite implantable device comprising a cage component and bone graft component.

FIGS. 8A and 8B are photographs showing top-down views of composite implantable devices comprising a metal cage component and a bone graft component packed and overfilled into the cage component.

FIG. 9 shows an implantable device formed of bone graft material.

FIG. 10 is a photograph of an implantable device formed of bioactive glass fibers.

FIG. 11 is a photograph of an implantable device formed of bioactive glass fibers randomly arranged and bioactive glass fibers aligned as bundles therein.

FIG. 12 is a photograph of an implantable device formed of aligned bioactive glass fibers.

The foregoing and other features of the present disclosure will become apparent to one skilled in the art to which the present disclosure relates upon consideration of the following description of exemplary embodiments with reference to the accompanying drawings.

### DETAILED DESCRIPTION

The present disclosure provides various implantable devices that comprise an improved bone graft material, such as for example, bioactive glass, or implantable devices that work in conjunction with an improved bone graft material and act as a composite implantable device, for the improved treatment of bone. These devices are bioactive, and are engineered to provide enhanced cellular activity to promote bone fusion or regrowth.

Characteristics of the Implantable Devices

The implantable devices of the present disclosure can generally be categorized as either a self-contained, or standalone, implantable device that is formed of an improved bone graft material, such as for example, bioactive glass, or a composite implantable device having a core framework that works in conjunction with an improved bone graft material. These devices may share similar shapes, structural and biochemical features, materials and clinical applications. Accordingly, the following descriptions regarding device properties and material properties are considered applicable to either category of implantable device.

The standalone implantable devices may be formed partially or entirely of the bone graft material, and may be load-bearing or non-load-bearing. The composite implantable devices of the present disclosure comprise a first, interbody fusion cage component and a second, bone graft material component. The two components work in synchrony to produce an overall improved bone fusion device. The composite implantable devices may be load bearing, or non-load bearing devices. The devices may be partially or fully resorbable. The devices may be applicable for use in all areas of the body, such as for example without limitation, the spine, shoulder, wrist, hip, knee, ankle, or sternum, as well as other joints like finger and toe joints. Other anatomical regions that can utilize this technology include the maxillofacial region, such as the jaw or cheeks, as well as the skull region. The devices may be shaped and sized to accommodate the specific anatomical region to which it is being applied. In the case of the spine, the spinal fusion device may be one of a PLIF, TLIF, CIF, ALIF, LLIF or OLIF cage, or a vertebral replacement device. The device may also be wedge shaped. The spinal fusion device may be inserted into a patient's intervertebral disc space for restoring disc height to the spinal column.

Furthermore, the composite implantable devices may be constructed to provide a connected pathway that directs the growth of bone. For instances, channels or porous networks in both the cage and the bone graft component may communicate with one another to allow true interconnectivity and synchrony during the fusion process.

Characteristics of the Cage

The interbody fusion cages of the present disclosure may be external to the bone graft component. However, in some examples, the interbody fusion cages are internal to the bone graft component. The cages may be formed of metal or polymer, or a combination of both. The cages may comprise a unitary body, or have interconnecting or interlocking components. In addition, the cages may contain one or more chambers for receiving the bone graft component.

The cages may also utilize 3D printing technology, or SLM (selective laser melting) technology, a form of layer-by-layer deposition process for highly customized implant production.

Characteristics of the Bone Graft Component

The materials of the bone graft component of the composite implantable devices are engineered with a combination of structural and functional features that act in synergy to allow the implant to support cell proliferation and new tissue growth over time. The bone graft components provide the necessary porosity and pore size distribution to allow proper vascularization, optimized cell attachment, migration, proliferation, and differentiation. The components are formed of synthetic materials that are biocompatible and offer the requisite mechanical integrity to support continued cell proliferation throughout the healing process.

The bone graft components may be formed of a synthetic material that is both biocompatible and bioabsorbable or bioresorbable. In addition, the synthetic material may be bioactive. In one embodiment, the material may be a material that is bioactive and forms a calcium phosphate layer on its surface upon implantation. In another embodiment, the material may comprise a bioactive glass (“BAG”). Suitable bioactive glasses include sol gel derived bioactive glass, melt derived bioactive glass, silica based bioactive glass, silica free bioactive glass such as borate based bioactive glass and phosphate based bioactive glass, crystallized bioactive glass (either partially or wholly), and bioactive glass containing trace elements or metals such as copper, zinc, strontium, magnesium, zinc, fluoride, mineralogical calcium sources, and the like. Examples of sol gel derived bioactive glass include S70C30 characterized by the general implant of 70 mol % SiO₂, 30 mol % CaO. Examples of melt derived bioactive glass include 45S5 characterized by the general implant of 46.1 mol % SiO₂, 26.9 mol % CaO, 24.4 mol % Na₂O and 2.5 mol % P₂O₅, S53P4, and 58S characterized by the general implant of 60 mol % SiO₂, 36 mol % CaO and 4 mol % P₂O₅. Another suitable bioactive glass may also be 13-93 bioactive glass.

The bioactive glass may form the base material from which the engineered bone graft components of the present disclosure are composed. The bioactive glass may take the form of fibers, granules, or a combination of both. By the term granules, what is meant is at least one fragment or more of material having a non-rod shaped form, such as a rounded, spherical, globular, or irregular body.

The bioactive glass may be provided in a materially pure form. Additionally, the bioactive glass may be mixed with a carrier for better clinical handling, such as to make a putty or foam material. A pliable material in the form of a putty may be provided by mixing the bioactive glass with a flowable or viscous carrier. A foam material may be provided by embedding the bioactive glass in a porous matrix such as collagen (either human or animal derived) or porous polymer matrix. One of the advantages of a foam material is that the porous carrier can also act as a site for attaching cells and growth factors, and may lead to a better managed healing.

The carrier material may be porous and may help contribute to healing. For example, the carrier material may have the appropriate porosity to create a capillary effect to bring in cells and/or nutrients to the implantation site. The carrier material may also possess the chemistry to create osmotic or swelling pressure to bring in nutrients to the site and resorb quickly in the process. For instance, the carrier material may be a polyethylene glycol (PEG) which has a high affinity to water.

In some cases, a dry matrix of bioactive glass granules and microspheres can be mixed with polymers such as collagen, polyethylene glycol, poly lactic acid, polylactic-glycolic acid, poly caprolactone, polypropylene-polyalkylene oxide co-polymers; with polysaccharides such as carboxymethy cellulose, hydroxypropyl methyl cellulose, with glycosaminoglycan such as hyaluronic acid, chondroitin sulfate, chitosan, N-acetyl-D-glucosamine, or with alginates such as sodium alginate. The dry matrix when hydrated and mixed forms a putty that can be used as mixed, or the product can be loaded into a syringe with a threaded plunger and delivered percutaneously. Alternately, the product can be mixed inside the syringe and delivered percutaneously to form the implantable device in situ.

The bioactive glass may be manufactured by electrospinning, or by laser spinning for uniformity. For example, where the material is desired in a fibrous form, laser spinning would produce fibers of uniform diameters. Further, the bioactive glass fibers may be formed having varying diameters and/or cross-sectional shapes, and may even be drawn as hollow tubes. Additionally, the fibers may be meshed, woven, intertangled and the like for provision into a wide variety of shapes.

The bone graft components may be engineered with fibers having varying resorption rates. The resorption rate of a fiber is determined or controlled by its material composition and by its diameter. The material composition may result in a slow reacting vs. faster reacting product. Similarly, smaller diameter fibers can resorb faster than larger diameter fibers of the same implant. Also, the overall porosity of the material can affect resorption rate. Materials possessing a higher porosity mean there is less material for cells to remove. Conversely, materials possessing a lower porosity mean cells have to do more work, and resorption is slower. Accordingly, the bone graft components may contain fibers that have the appropriate material composition as well as diameter for optimal performance. A combination of different fibers may be included in the component in order to achieve the desired result.

Equally as important as the material composition and diameter is the pore size distribution of the open porosity and in particular the surface area of the open porosity. The present bone graft components provide not only an improved pore size distribution over other bone graft materials, but a higher surface area for the open pores. The larger surface area of the open porosity of the present implants drives faster resorption by body fluids, allowing the fluid better access to the pores.

Similar to the bioactive glass fibers, the inclusion of bioactive glass granules can be accomplished using particulates having a wide range of sizes or configurations to include roughened surfaces, very large surface areas, and the like. For example, granules may be tailored to include interior lumens with perforations to permit exposure of the surface of the granule's interior. Such granules would be more quickly absorbed, allowing a tailored implant characterized by differential resorbability. The perforated or porous granules could be characterized by uniform diameters or uniform perforation sizes, for example. The porosity provided by the granules may be viewed as a secondary range of porosity accorded the devices. By varying the size, transverse diameter, surface texture, and configurations of the bioactive glass fibers and granules, if included, the manufacturer has the ability to provide a bioactive glass bone graft material with selectively variable characteristics that can greatly affect the function of the implant before and after it is implanted in a patient. The nano and micro sized pores provide superb fluid soak and hold capacity, which enhances the bioactivity and accordingly the repair process.

Due to the pliability of this fibrous graft material, these same bioactive glass fibers may be formed or shaped into fibrous clusters with relative ease. These clusters can be achieved with a little mechanical agitation of the bioactive glass fibrous material. The resultant fibrous clusters are extremely porous and can easily wick up fluids or other nutrients. Hence, by providing the bioactive glass material in the form of a porous, fibrous cluster, even greater clinical results and better handling can be achieved.

The formed and shaped bioactive glass materials of the present disclosure, either with or without sintering, share similar attributes with a finite density material that has been dictated by its processing and the fiber dimensions of the base material (e.g., diameter and length of the fibers) that resulted in the cluster formation. The ultra-porous clusters can possess nano, micro, meso, and macro porosity in a gradient throughout the cluster. Without limitation, a nanopore is intended to represent a pore having a diameter below about 1 micron and as small as 100 nanometers or smaller, a micropore is intended to represent a pore having a diameter between about 1 to 10 microns, a mesopore is intended to represent a pore having a diameter between about 10 to 100 microns, and a macropore is intended to represent a pore having a diameter greater than about 100 microns and as large as 1 mm or even larger. Under a consistent manufacturing process, the formed clusters of bioactive glass can be used with volumetric dosage to fill a bone defect. Any number of differently sized clusters can be provided for various clinical applications.

One of the benefits of providing an ultra-porous bioactive glass material in cluster form is that handling of the material can be improved. In one manner of handling the cluster of materials, the clusters may be packaged in a syringe with a carrier, and injected into the fusion cage or directly into the bone defect with ease. Another benefit is the additional structural effect of having a plurality clusters of fibers closely packed together, forming additional macrostructures to the overall scaffold of material. Like a sieve, the openings between individual clusters can be beneficial such as when a filter is desired for various nutrients in blood or bone marrow to concentrate certain desired nutrients at the implant location.

Of course, it is understood that, while the term cluster is used to describe the shape of the materials, such term is not intended to limit the invention to spherical shapes. In fact, the formed cluster shape may comprise any rounded or irregular shape, so long as it is not a rod shape. In the present disclosure, the term fibrous cluster represents a matrix of randomly oriented fibers of a range of sizes and length. Additional granules or particulates of material may be placed randomly inside this matrix to provide additional advantages. A variety of materials and structure can optionally be employed to control the rate of resorption, osteostimulation, osteogenesis, compression resistance, radiopacity, antimicrobial activity, rate of drug elution, and provide optimal clinical handling for a particular application.

The use of fused or hardened fiber clusters may be advantageous in some instances, because the fusing provides relative hardness to the clusters, thereby rendering the hardened clusters mechanically stronger. Their combination with the glass granules further enhances the structural integrity, mechanical strength, and durability of the implant. Because larger sized granules or clusters will tend to have longer resorption time, in previous cases the user had to sacrifice strength for speed. However, it is possible to provide larger sized granules or clusters to achieve mechanical strength, without significantly sacrificing the speed of resorption. To this end, ultra-porous clusters can be utilized as just described for fiber-based and glass-based clusters. Rather than using solid spheres or clusters, the present disclosure provides ultra-porous clusters that have the integrity that overall larger sized clusters provide, along with the porosity that allows for speed in resorption. These ultra-porous clusters will tend to absorb more nutrients, resorb quicker, and lead to much faster healing and remodeling of the defect.

In some embodiments, the fiber clusters may be partially or fully fused or hardened to provide hard clusters. Of course, it is contemplated that a combination of both fused fiber clusters (hard clusters) and unfused or loose fiber clusters (soft clusters) may be used in one application simultaneously. Likewise, combinations of putty, foam, clusters and other formulations of the fibrous graft material may be used in a single application to create an even more sophisticated porosity gradient and ultimately offer a better healing response. In some cases, solid porous granules of the bioactive glass material may also be incorporated into the implant.

As previously discussed, the ideal bone graft material must possess a combination of features that act in synergy to allow the bone graft material to support the biological activity of tissue growth and mechanism of action as time progresses. It is known that porosities and pore size distribution play a critical role in the clinical success of bone graft materials. More specifically, the bone graft material needs to include an appropriate pore size distribution to provide optimized cell attachment, migration, proliferation and differentiation, and to allow flow transport of nutrients and metabolic waste. In addition, in a porous structure the amount and size of the pores, which collectively form the pore size gradient, will be directly related to the mechanical integrity of the material as well as affect its resorption rate. Having a stratified porosity gradient will provide a more complex resorption profile for the bone graft component of the devices, and engineering the material with a suitable pore size gradient will avoid a resorption rate that is too fast or too slow.

Desirably, pore size distribution includes a range of porosities that includes macro, meso, micro and nano pores. As previously mentioned, without limitation, a nanopore is intended to represent a pore having a diameter below about 1 micron and as small as 100 nanometers or smaller, a micropore is intended to represent a pore having a diameter between about 1 to 10 microns, a mesopore is intended to represent a pore having a diameter between about 10 to 100 microns, and a macropore is intended to represent a pore having a diameter greater than about 100 microns and as large as 1 mm or even larger. Accordingly, the bioactive glass material may be provided with variable degrees of porosity, and is preferably ultraporous. In one embodiment, the material may have a range of porosities including macro, meso, micro and nano pores. The resultant engineered implant may also include the same range of porosities, which could be provided as a porous network of matrices within the fibrous scaffold and around the material. Accordingly, porosity may be provided inherently by the actual bioactive glass material itself, as well as the matrices separating the material within the overall component.

Another feature of the engineered bone graft materials of the present disclosure is their ability to provide mechanical integrity to support new tissue growth. Not only should the bone graft component provide the appropriate biocompatibility and resorption rate, but the surface area should be maximized to fully support cell proliferation. The engineered component can be selectively composed and structured to have differential or staged resorption capacity, while still being easily molded or shaped into clinically relevant shapes as needed for different surgical and anatomical applications. Additionally, these engineered components may have differential bioresorbability, compression resistance and radiopacity, and can also maximize the content of active ingredient relative to carrier materials such as for example collagen.

The bone graft components formed from these materials are able to sustain tissue growth throughout the healing process. One of the deficiencies of currently available bone graft materials is their lack of ability to provide proper mechanical scaffolding while supporting cell proliferation over time. The engineered materials and implants of the present disclosure overcome this problem by providing, among other things, an appropriate combination of porosities (i.e., pore size distribution) and high surface area within a porous bioactive glass infrastructure that serves as an ideal scaffold for tissue growth. More importantly, the range of porosities is distributed throughout the porous bioactive glass infrastructure, which is able to support continued cell proliferation throughout the healing process.

Initially upon implantation, the engineered implants provide a network of macro, meso, micro and nano pores distributed within a fibrous bioactive glass matrix. These pores can be interconnected, allowing cell migration throughout the matrix. As surface area is inversely proportional to the diameter of the pore, the engineered implants maximize surface area for cell attachment by providing a desired surface-to-volume ratio of nano sized pores. The laws of physics suggest that these smaller pores are optimal for vascularization. Due to the osmotic pressure of the environment, a capillary effect will be observed with the nano and micro sized pores that result in biological fluid being wicked towards the center of the bioactive glass matrix. Likewise, the larger pores like the macro sized pores are optimal for oxygenation and nutrient exchange within the matrix.

After implantation, a calcium phosphate (CaP) layer forms around the construct. This calcium phosphate layer results from the chemical interaction of the bioactive glass material and the surrounding biological environment. At the same time, the smaller sized pores like the nano sized pores will be resorbing at a rate faster than the rest of the implant. As these nano sized pores resorb or become replaced with cells, they will bring in cellular activity and create a three-dimensional biostructure that, within itself, also has its own porosity. Thus, over time, new cells replace the resorbed material at a rate that maintains the mechanical integrity of the new construct. The new cells form their own network around the fibrous bioactive glass matrix, which fibers provide connectivity for the tissue growth. More importantly, because of the widespread distribution of nanopores throughout the fibrous matrix, the new cells are present in a density that makes the implant mechanically sound.

Unlike traditional bone graft scaffolds, the present bone graft materials offer both the necessary structure and function for clinical success, and allow the process of cell proliferation to occur in a non-uniform, multi-faceted fashion with the appropriate balanced rate of new cell proliferation replacing resorbed graft material. More importantly, this replacement occurs at select locations within the construct, without compromising overall mechanical integrity. In addition, the materials and implants allow this new tissue growth process to occur throughout the healing process, not just at the beginning of the process. The constant and simultaneous activities of cell proliferation and resorption occur throughout the entire healing time with the present bone graft materials and implants.

In some embodiments, the underlying bioactive material forming the foundation of the implant may be a bioactive glass. The bioactive glass may take the form of fibers, making them easy to handle in a clinical setting. Accordingly, in one embodiment, the engineered implant may be a fibrous scaffold formed of fibrous bioactive glass fibers. These fibers may be unrestricted, and allowed to move freely over one another. Alternatively, the fibers may be partially or fully fused to provide a more organized, rigid and structured network of fibers. Such a fibrous scaffold would allow for stimulation and induction of the natural biologic healing process found in fibrin clots whose mechanism is similar to that of new bone formation. One theory of the mechanism of action as provided by the fibrous nature of the scaffold is provided below.

The standard method for healing natural tissue with synthetic materials has been to provide a device having the microstructure and macrostructure of the desired end product. Where the desired end product is cancellous bone, traditional bone grafts have been engineered to mimic the architecture of cancellous bone. Although this has been the current standard for bone grafts, it does not take into account the fact that bone is a living tissue. Each bony trabeculae is constantly undergoing active biologic remodeling in response to load, stress and/or damage. In addition, cancellous and cortical bone can support a vast network of vasculature. This network not only delivers nutrients to sustain the living environment surrounding bone, but also supports red blood cells and marrow required for basic biologic function. Therefore, merely providing a synthetic material with the same architecture that is non-biologic is insufficient for optimal bone healing and bone health. Instead, what is required is a mechanism that can recreate the living structure of bone.

Traditional synthetics act as a cast, or template, for normal bone tissue to organize and form. Since these synthetics are not naturally occurring, eventually the casts or templates have to be resorbed to allow for normal bone to be developed. If these architectured synthetics do not resorb and do not allow proper bone healing, they simply become foreign bodies that are not only obstacles, but potentially detrimental, to bone healing. This phenomenon has been observed in many studies with slow resorbing or non-resorbing synthetics. Since these synthetics are just chemically inert, non-biologic structures that only resemble bone, they behave as a mechanical block to normal bone healing and development.

With the understanding that bone is a living biologic tissue and that inert structures will only impede bone healing, a different physiologic approach is presented with the present invention. Healing is a phasic process starting with some initial reaction. Each phase builds on the reaction that occurred in the prior phase. Only after a cascade of phases does the final development of the end product occur—new bone tissue. The traditional method has been to replace or somehow stimulate healing by placing an inert final product as a catalyst to the healing process. This premature act certainly does not account for the physiologic process of bone development and healing.

The physiologic process of bone healing can be broken down to three phases: (a) inflammation; (b) osteogenesis; and (c) remodeling. Inflammation is the first reaction to injury and a natural catalyst by providing the chemotactic factors that will initiate the healing process. Osteogenesis is the next phase where osteoblasts respond and start creating osteoid, the basic material of bone. Remodeling is the final phase in which osteoclasts and osteocytes then recreate the three-dimensional architecture of bone.

In a normal tissue repair process, at the initial phase a fibrin clot is made that provides a fibrous architecture for cells to adhere. This is the cornerstone of all connective tissue healing. It is this fibrous architecture that allows for direct cell attachment and connectivity between cells. Ultimately, the goal is to stimulate cell proliferation and osteogenesis in the early healing phase and then allow for physiologic remodeling to take place. Since the desired end product is living tissue, the primary objective is to stimulate as much living bone as possible by enhancing the natural fiber network involved in initiation and osteogenesis as well as angiogenesis.

Fibrous bone graft components formed from these fibrous materials attempt to recapitulate the normal physiologic healing process by presenting the fibrous structure of the fibrin clot. Since these bioactive implants made of fibers are both osteoconductive as well as osteostimulative, the fibrous network will further enhance and accelerate bone induction. Further, the free-flowing nature of the bioactive fibrous matrix or scaffold allows for natural initiation and stimulation of bone formation rather than placing a rigid template that may impede final formation as with current graft materials. The fibers of the implants can also be engineered to provide a chemical reaction known to selectively stimulate osteoblast proliferation or other cellular phenotypes.

The present disclosure provides several embodiments of fibrous bone graft materials formed of bioactive glass fibers. The bundles of bioactive glass fibers are ultraporous, and include a combination of nano, micro, meso and macro pores. The fibrous nature of the material allows the bioactive glass fibers to be easily molded or shaped into clinically relevant shapes as needed for different surgical and anatomical applications, while maintaining the material's porosity. One manner of molding or shaping the scaffold is by placing the fibers into a mold tray. The implant may comprise bioactive glass fibers alone, or with additives as described above.

Due to the fibrous and pliable nature of the base material, it is also possible to add a biological fluid to the fibrous matrix and press into a formed shape with the fluid contained therein. Of course, it is understood that the fibrous material may just as easily be compressed in a mold. Liquids like bone marrow aspirate, glue or other binding agents may be added to the material prior to molding. In addition, a solvent exchange may be utilized and the shaped material can be allowed to dry or cure to form a hardened solid scaffold for implantation.

The fibers forming the component have a relatively small diameter, and in particular, a diameter in the range of about 500 nanometers to about 50 microns, or a diameter in the range of about 0.1 to about 100 microns. In one embodiment, the fiber diameter can be less than about 10 nanometers, and in another embodiment, the fiber diameter can be about 5 nanometers. In some embodiments, the fiber diameter can be in the range of about 0.5 to about 30 microns. In other embodiments, the fiber diameter can fall within the range of between about 2 to about 10 microns. In still another embodiment, the fiber diameter can fall within the range of between about 3 to about 4 microns.

The bioactive glass fibers may be manufactured having predetermined cross-sectional diameters as desired. In one example, the bone graft material may be formed from a randomly oriented matrix of fibers of uniform diameters. Further, the bioactive glass fibers may be formed having varying diameters and/or cross-sectional shapes, and may even be drawn as hollow tubes. Additionally, the fibers may be meshed, woven, intertangled and the like for provision into a wide variety of shapes.

For example, a bioactive glass fiber component can be manufactured such that each fiber is juxtaposed or out of alignment with the other fibers could result in a randomly oriented fibrous matrix appearance due to the large amount of empty space created by the random relationship of the individual glass fibers within the material. Such an implant easily lends itself to incorporating additives randomly dispersed throughout the fibers, such as those previously described and including bioactive glass granules, antimicrobial fibers, particulate medicines, trace elements or metals such as copper, which is a highly angiogenic metal, strontium, magnesium, zinc, etc. mineralogical calcium sources, and the like. Further, the bioactive glass fibers may also be coated with organic acids (such as formic acid, hyaluronic acid, or the like), mineralogical calcium sources (such as tricalcium phosphate, hydroxyapatite, calcium carbonate, calcium hydroxide, calcium sulfate, or the like), antimicrobials, antivirals, vitamins, x-ray opacifiers, or other such materials.

The component may be engineered with fibers having varying resorption rates. The resorption rate of a fiber is determined or controlled by, among other things, its material composition and by its diameter. The material composition may result in a slow reacting vs. faster reacting product. Similarly, smaller diameter fibers can resorb faster than larger diameter fibers. Also, the overall porosity of the material can affect resorption rate. Materials possessing a higher porosity mean there is less material for cells to remove. Conversely, materials possessing a lower porosity mean cells have to do more work, and resorption is slower. Accordingly, the component may contain fibers that have the appropriate material composition as well as diameter for optimal performance. A combination of different fibers may be included in the construct in order to achieve the desired result. For instance, the implant may comprise a composite of two or more fibers of a different material, where the mean diameter of the fibers of each of the materials could be the same or different.

Equally as important as the material composition and diameter is the pore size distribution of the open porosity and in particular the surface area of the open porosity. The present bone graft materials provide not only an improved pore size distribution over other bone graft materials, but a higher surface area for the open pores. The larger surface area of the open porosity of the present implants drives faster resorption by body fluids, allowing the fluid better access to the pores.

Another manner of further enhancing the bioactive graft material of the present disclosure is to provide an additional layer or coating of polymer over the material in its individual fiber form or in its shaped fibrous cluster form. For example, biocompatible, bioabsorbable polymer or film-forming agents such as polycaprolactones (PCL), polyglycolic acid (PGA), poly-L-Lactic acid (PL-LA), polysulfones, polyolefins, polyvinyl alcohol (PVA), polyalkenoics, polyacrylic acids (PAA), PEG, PLGA, polyesters and the like are suitable materials for coating or binding the fibrous graft material of the present invention. The resultant product is strong, carveable, and compressible, and may still absorb blood. Other suitable materials also include artificial polymers selected from poly(anhydrides), poly(hydroxy acids), polyesters, poly(orthoesters), polycarbonates, poly(propylene fumerates), poly(caprolactones), polyam ides, polyamino acids, polyacetals, polylactides, polyglycolides, polysulfones, poly(dioxanones), polyhydroxybutyrates, polyhydroxyvalyrates, poly(vinyl pyrrolidones), biodegradable polycyanoacrylates, biodegradable polyurethanes, polysaccharides, tyrosine-based polymers, poly(methyl vinyl ether), poly(maleic anhydride), poly(glyconates), polyphosphazines, poly(esteram ides), polyketals, poly(orthocarbonates), poly(maleic acid), poly(alkylene oxalates), poly(alkylene succinates), poly(pyrrole), poly(aniline), poly(thiophene), polystyrene, non-biodegradable polyurethanes, polyureas, poly(ethylene vinyl acetate), polypropylene, polymethacrylate, polyethylene, poly(ethylene oxide), and co-polymers, adducts, and mixtures thereof. The material may be partially or fully water soluble.

Applying this feature to the fibrous graft material of the present disclosure, in one embodiment the individual fibers of the bioactive glass fiber material may be coated with such a biocompatible polymer. The coating itself would be sufficiently thin so as not to impede the advantages from the physical attributes and bioactive properties of the base material as described. In other words, the polymeric coated fibers would still retain pliability and allow the user to easily mold or form the fibrous material into the desired shape for implantation. Such a polymeric coating would further enhance the handling of the fibrous material while still allowing the underlying base material to be used in the same manner as previously described. The polymeric component would also provide a mechanism for graft containment, controlled resorption, and controlled bioactivity or cellular activity. This polymeric component may comprise a solid layer, a porous or perforated layer, or a mesh or woven layer of material having channels therein for exchange of nutrients, cells or other factors contained within.

In another embodiment, the fibrous graft material may be formed or shaped into an initial geometry and then coated with the biocompatible polymer. For example, the fibrous graft material may be formed into fibrous clusters as previously mentioned. These fibrous clusters can then be encapsulated in a biocompatible polymer. The resulting implant would have a fibrous BAG center surrounding which is a polymeric coating or shell.

Bioactive materials of the invention may be prepared using electrospinning techniques. Electrospinning uses an electrical charge to draw very fine (typically on the micro or nano scale) fibers from a liquid or a slurry. When a sufficiently high voltage is applied to a liquid droplet, the body of the liquid becomes charged. The electrostatic repulsion in the droplet would counteract the surface tension and the droplet is stretched. When the repulsion force exceeds the surface tension, a stream of liquid erupts from the surface. This point of eruption is known as a Taylor cone. If molecular cohesion of the liquid is sufficiently high, the stream does not breakup and a charged liquid jet is formed. As the jet dries in flight, the mode of current flow changes from ohmic to convective as the charge migrates to the surface of the fiber. The jet is then elongated by a whipping process caused by electrostatic repulsion initiated at small bends in the fiber, until it is finally deposited on a grounded collector. The elongation and thinning of the fiber resulting from this bending instability leads to the formation of uniform fibers with nanometer-scale diameters.

While the voltage is normally applied to the solution or slurry in a regular electrospinning process, according to embodiments of the present invention, the voltage is applied to the collector, not to the polymer solution (or slurry), and, therefore, the polymer solution is grounded. The polymer solution or slurry is sprayed into fibers while applying the voltage in this manner, and the fibers are entangled to form a three-dimensional structure.

The biocompatible polymeric coating may be heat wrapped or heat shrunk around the underlying fibrous bone graft material. In addition, the biocompatible polymeric coating may be a mixture of polymer and other components. For example, it is contemplated that the polymeric coating can comprise 100% of a particular polymer, such as for instance, PLA. However, a mixture of 50% PLA and 50% PEG may also be utilized. Likewise, the coating may be formed of a polymer—BAG composition. In this case, the coating could comprise 50% polymer with the remaining 50% comprising BAG granules or fibers, for instance. Of course, it is understood that the percentage of an individual component may vary as so desired, and the percentages provided herein are merely exemplary for purposes of conveying the concept.

An alternative material suitable for binding or containing the fibrous graft material is collagen, which could be provided as a slurry and then hardened such as by freeze-drying. This collagen could be human-derived collagen or animal-derived collagen, for instance.

Additionally, it is contemplated that additional BAG granules, beads, spheres, etc. or individual fibers may be adhered to the polymeric coating in order to provide a surface enhancement for adherence to the implant site. These BAG granules or fibers would allow a better friction fit with the patient, serving as structural features. For example, added surface features may include fibers, granules, particulates, and the like that can be included in the coating to provide an exterior with bioactive anchorage points to attract cellular activity and improve adhesion of the implant in situ.

At the same time, these additional BAG granules or fibers also serve as bioactive features to allow for a differential mechanism of resorption and a more sophisticated bioactivity profile, since these BAG granules and fibers are themselves also capable of initiating bioactivity. The BAG granules or fibers may be used with or without additional coatings, such as with or without the additional polymer coating. Moreover, it is understood that part or all of the BAG fibers and materials may be sintered or unsintered in these applications.

The addition of the polymeric component to the base fiber graft material provides the benefit of allowing ease of handling, but also adds a layer of control to the resorption rate and bioactivity. It could easily be contemplated that the polymeric component in all of the embodiments previously described could be porous itself, thereby providing a composite implant having controlled fluid interactivity. The ability to provide separate layers of BAG within a single implant also renders depth control to the bioactivity, as well as controlled graft containment.

The embodiments of the present disclosure are not limited, however, to fibers alone. In other embodiments, the bioactive glass fibers that form the foundation of the implant may be substituted or supplemented with bioactive granules. These granules may be uniform or non-uniform in diameter, and may comprise a mixture of differently sized diameters of granules. In addition, the granules may be formed of the same type of bioactive glass material, or a mixture of different materials selected from the group of suitable materials previously mentioned. The granules may be solid or porous, and in some cases a mixture of both solid and porous granules may be used. Regardless, the engineered implant comprising the granular foundation should still provide the desired pore size distribution, which includes a range of porosities that includes macro, meso, micro and nano pores.

Like the fibers, at least some or all of the granules forming the engineered implant may be coated with a polymeric coating. The coating may be solid or porous. This coating could be provided on individual granules, or it could envelope a cluster or group of granules. In other embodiments, the coating could comprise collagen or hydroxyapatite (HA). For instance, the coating could be a solid collagen or a perforated collagen. Added surface features including fibers, granules, particulates, and the like can be included in the coating to provide an exterior with bioactive anchorage points to attract cellular activity and improve adhesion of the implant in situ. In addition the surface features may also serve to initiate bioactivity by creating debridement at the area of insertion.

In addition, some embodiments may include a mixture of both granular bioactive glass as the primary material with secondary bioactive glass fibers as the carrier material. In such cases, both the primary and secondary materials are active. The fibrous carrier would be able to resorb quickly to create a chemically rich environment for inducing new cellular activity. Moreover, the fibrous material would serve as select attachment or anchorage sites for bone forming cells.

In some embodiments, at least some or all of the engineered implant may be coated with a glass, glass-ceramic, or ceramic coating. The coating may be solid or porous, and provide for better handling of the fibrous bioactive glass material. In one embodiment, the coating may be a bioactive glass such as 45S5 or S53P4. In another embodiment, the coating may be partially or fully fused such as by an application of high heat to melt some of the fibrous material, creating a slightly hardened or fully fused shell of material. For instance, this fusing or hardening would lead to a semi-soft crust, while the full sintering would lead to a hard crust around some or all of the implant.

In still further embodiments, the implants may comprise a multi-layered composite of varying or alternating materials. For example, in one case a bioactive glass fiber or granule may be encased in a polymer as described above, and then further encased in a bioactive glass. This additional bioactive glass layer could be the same as, or different, than the underlying bioactive glass. The resultant construct would therefore have varying resorption rates as dictated by the different layers of materials.

In addition to providing a structurally sound implant and the appropriate materials and porosities and pore size gradient for cell proliferation, the present bone graft materials and implants may also provide cell signals. This can be accomplished by the incorporation of biological agents such as growth factors. These factors may be synthetic, recombinant, or allogenic, and can include, for example, stem cells, demineralized bone matrix (DBM), as well as other known cell signaling agents.

In some embodiments, the engineered implants may be also osteoconductive and/or osteostimulatory. By varying the diameter and chemical composition of the components used in the embodiments, the engineered implants may have differential activation (i.e., resorbability), which may facilitate advanced functions like drug delivery of such drugs as antibiotics, as an example. One manner of providing osteostimulative properties to the implant is to incorporate bone marrow into the fibrous matrix. The incorporation of the marrow would produce an osteostimulative implant that accelerates cell proliferation.

In other embodiments, the engineered implant may also include trace elements or metals such as copper, zinc, strontium, magnesium, zinc, fluoride, mineralogical calcium sources, and the like. These trace elements provide selective benefits to the engineered structural and functioning implants of the present disclosure. For example, the addition of these trace elements like strontium may increase x-ray opacity, while the addition of copper provides particularly effective angiogenic characteristics to the implant. The materials may also be coated with organic acids (such as formic acid, hyaluronic acid, or the like), mineralogical calcium sources (such as tricalcium phosphate, hydroxyapatite, calcium sulfate, calcium carbonate, calcium hydroxide, or the like), antimicrobials, antivirals, vitamins, x-ray opacifiers, or other such materials. These bone graft materials may also possess antimicrobial properties as well as allow for drug delivery. For example, sodium or silver may be added to provide antimicrobial features. In one embodiment, a layer or coating of silver may be provided around the engineered implant to provide an immediate antimicrobial benefit over an extensive surface area of the implant. Other suitable metals that could be added include gold, platinum, indium, rhodium, and palladium. These metals may be in the form of nanoparticles that can resorb over time.

Additionally, biological agents may be added to the engineered material. These biological agents may comprise bone morphogenic protein (BMP), a peptide, a bone growth factor such as platelet derived growth factor (PDGF), vascular endothelial growth factor (VEGF), insulin derived growth factor (IDGF), a keratinocyte derived growth factor (KDGF), or a fibroblast derived growth factor (FDGF), stem cells, bone marrow, and platelet rich plasma (PRP), to name a few. Other medicines may be incorporated into the scaffold as well, such as in granular or fiber form. In some cases, the bone graft material serves as a carrier for the biological agent, such as BMP or a drug, for example.

Embodiments of the present disclosure may be explained and illustrated with reference to the drawings and photographs. It should be understood, however, that the drawings are not drawn to scale, and are not intended to represent absolute dimensions or relative size. Rather, the drawings and photographs help to illustrate the concepts described herein.

### EXAMPLES OF COMPOSITE IMPLANTABLE DEVICES

The following are examples of load bearing devices where the bioactive glass (BAG) material is the sole or primary component of the implantable device. By load bearing, what is meant is a device having about 50 mPa compression strength and about 100 mPa tensile strength:

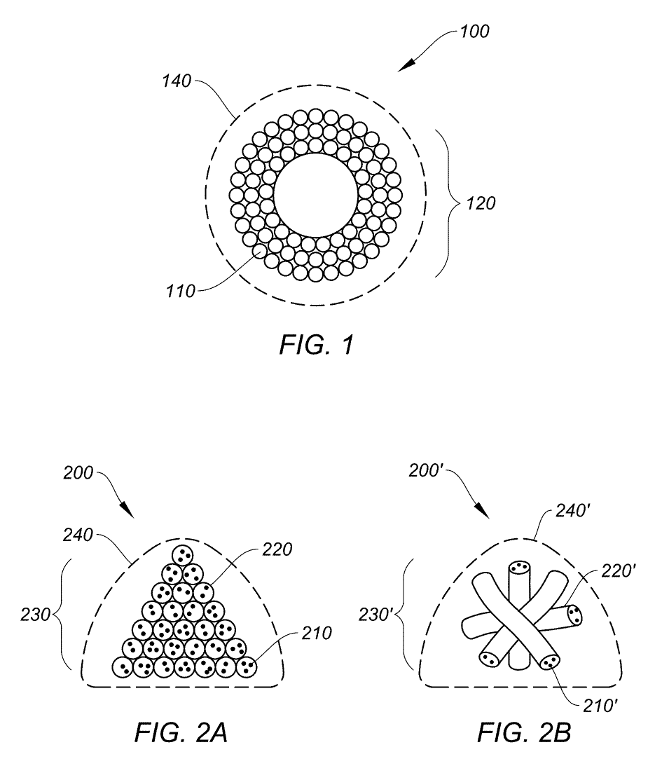

### Example 1—FIG. 1

The bone graft component of the composite implantable devices may be fibrous in nature, and comprise bioactive glass fibers. These fibers may be specifically aligned for directionality. In one example, as shown in FIG. 1, the composite implantable device 100 may comprise bundles 120 of individual fibers 110, with the fibers 110 being unidirectional within a particular bundle 120. A coating 140 may optionally be provided around the bundles 120. The bundles 120 may be arranged in a particular pattern, such as in a cylinder, as illustrated.

### Examples 2 and 3—FIGS. 2A and 2B

In other exemplary embodiments, the individual bundles may be selectively aligned, so as to provide an overall effect of purposeful directionality. For example, FIG. 2A shows a composite implantable device 200 in which a plurality of bundles 220 of individual fibers 210 are uniformly aligned, and which may optionally include a coating 240 surrounding the bundles 220. FIG. 2B shows a composite implantable device 220′ in which a plurality of bundles 220′ of individual fibers 210′ are randomly aligned to provide multidirectionality. The plurality of fibers 210, 210′ within each bundle 220, 220′ allow for robust cellular growth, while also controlling the directionality of the growth. An optional coating 240, 240′ may be provided for each device 200, 200′.

### Example 4

In one exemplary embodiment, the bone graft component may comprise morsels only. The morsels may be defined as an intricate nest of fibers and microspheres encased in a porous outer shell, all made of bioactive glass. These morsels may further be coated or encapsulated in a manner previously described, such as for example, with a glass or polymer.

### Example 5

In another exemplary embodiment, the bone graft component may comprise a combination of fibers and morsels. For instance, bioactive glass fibers may be wetted with a wetting agent like water to form a paste-like consistency. Then, bioactive glass morsels may be added to this paste to form a fiber-morsel mixture that may be added to the interbody cage component. This mixture may be packed or filled within the cage component, or it may surround the outside of the cage component such that the cage component is contained inside the mixture. Other alternative wetting agents include body fluids like blood or bone marrow aspirate, saline, or collagen.

### Example 6

In still another exemplary embodiment, the bone graft component may comprise hollow spheres that can carry an active agent for drug delivery, such as for BMP delivery. In one example, pretreated calcium phosphate may be used to provide better binding. In other examples, the hollow spheres may comprise hollow silicone microspheres. These silicone microspheres may further be resorbable, and/or pre-treated to get a HA/CO₃ outer layer. The microspheres provide the benefit of a high surface area with which to deliver the drug, allowing for better delivery.

In all examples above, some or all of the devices may be coated. For instance, individual fibers, bundles and/or morsels, or the entire collection of fibers, bundles and/or morsels may be coated or encapsulated in a manner previously described, such as for example, with a glass or polymer.

Further, the outer surface may be flash sintered or partially sintered to create a hardened outer surface or shell. Localized sintering allows for localized hardening, such that the user can control the areas where hardening is desired by selectively sintering that region of the device. This shell or crust may be porous, if so desired.

Additionally, the fibers of the above devices may be augmented with other materials for added support. These materials may include without limitation PEEK, BIS, PMMA, and others. The materials may be bioresorbable. Thus, it is possible to add various polymers to the underlying bioactive glass materials to create a differential resorption profile. For instance, a device in which the polymeric material may resorb faster than the BAG material would create open channels within the BAG fiber and/or morsel component to allow bone growth therethrough. By layering different bioactive materials, the device also provides staged or staggered bioactivity over time.

Another optional material to augment the BAG component is a degradable metal. One such metal may be a magnesium alloy. Still, other materials for augmentation include thermoplastics and/or thermoresins. Others still include calcium phosphates, citrates, gelatins, cellulose, or collagen.

Some of these augmentation materials may be radiopacifiers. Where the material is radiolucent, the BAG component may serve as a visual marker. For example, barium borosilicate may be contained in the glass component to allow the glass to act as a marker. To protect the polymer from the glass, the glass may be silanized in order to allow for wicking. Additionally, these devices may include surface features, such as for example, teeth, ridges, roughenings, short strands of fibers, particulates, and the like, to improve its adhesion properties. The surface features may additional serve to initiate biological activity by creating debridement at the site of insertion.

The following are examples of non-load bearing devices. These devices may include a cage component along with a bone graft component. The cage may be formed of temperature-sensitive material or non-temperature sensitive material. Further the cage may be external to the bone graft component, or the cage may be internally encapsulated within the bone graft component:

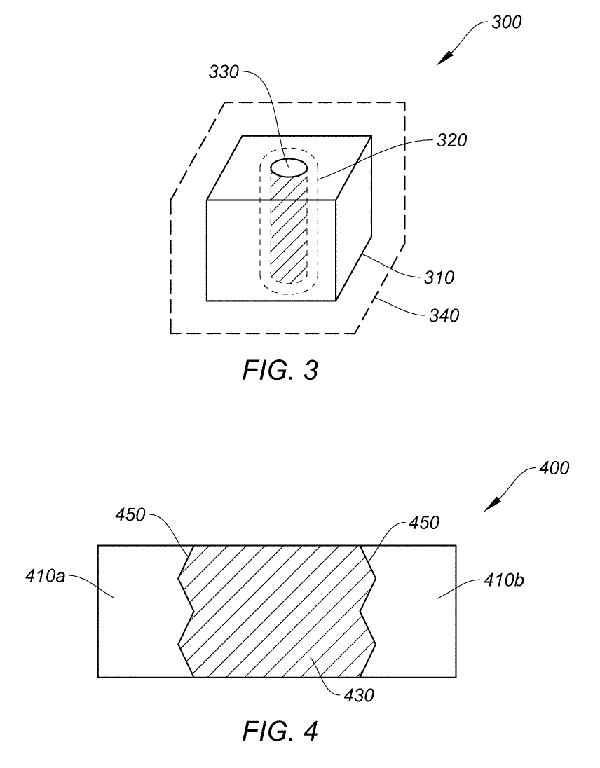

### Example 7—FIG. 3

In one exemplary embodiment, the cage component 310 of the composite implantable device 300 may be a PEEK (polyetheretherketone) cage, with PEEK being a temperature sensitive material. In its simplest form, the cage 310 may have a bone graft containment chamber 320 for receiving the bone graft component 330. As illustrated, in one embodiment, the containment chamber 320 may be filled with a plug 330 formed of bioactive glass. The plug 330 may comprise fibers, morsels, or any combination thereof. The fibers may also be aligned or not aligned, as described earlier. In other embodiments, this containment chamber 320 may be tapered to allow ease of packing material therein. The cage may have a wedge shape to facilitate its insertion. The cage may be pre-filled with the bone graft component and be encapsulated. For instance, the entire cage plus graft component may be coated or covered with a skin 340 of material such as those previously mentioned above. The coating or skin may or may not be porous. Further, surface features may be provided on the coating or skin.

Suitable filler material may include BAG fibers, BAG morsels, microspheres containing drugs or other active agents, or a collagen slurry, for instance. If desired, allograft material may be included. The allograft material may include bone chips, stem-cell preserved bone chips, or human-derived collagen. These package materials may also be pre-treated or wetted, such as with a solution like water, saline, blood, bone marrow aspirate, or other suitable fluids. Bone cement may also be used.

### Example 8

Alternatively, the component may be provided as a separate, pre-formed plug 330 that is inserted into the cage later, similar to the one shown in FIG. 3. This plug may be tapered in shape to match the tapered containment chamber. The plug may also include recesses to receive autograft or allograft material. Autograft material may be added to the plug in the OR, while allograft material may be added to the plug in its preformed state.

### Example 9

In another embodiment, the PEEK cage may have a bone graft containment chamber that is lined with BAG material. The liner of BAG material helps to secure the BAG pre-formed plug inside the PEEK cage, and acts as a gasket. This pre-formed plug has a shape that matches the containment chamber for a secure interference fit. Alternatively, the pre-formed plug may comprise a localized collagen plug.

### Example 10—FIG. 4

In still another embodiment, the composite implantable device 400 may comprise multiple interlocking components. For instance, the PEEK cage component(s) and bone graft component(s) may include shaped connection surfaces like threads, fins, a dovetail, tongue and groove, shark's tooth, and other similar structural features that allow for individual components to interlock onto one another. The PEEK component may be screwed onto, or slid onto, the bone graft component, for example, to form the composite device. In addition, the BAG component may comprise oriented fibers, morsels, or a combination of both. As shown, a bioactive glass main body 430 may have an interlocking end that allows caps 410a, 410b to lock on at these interlocking junctions 450. These caps 410a, 410b may be formed of PEEK, for example.

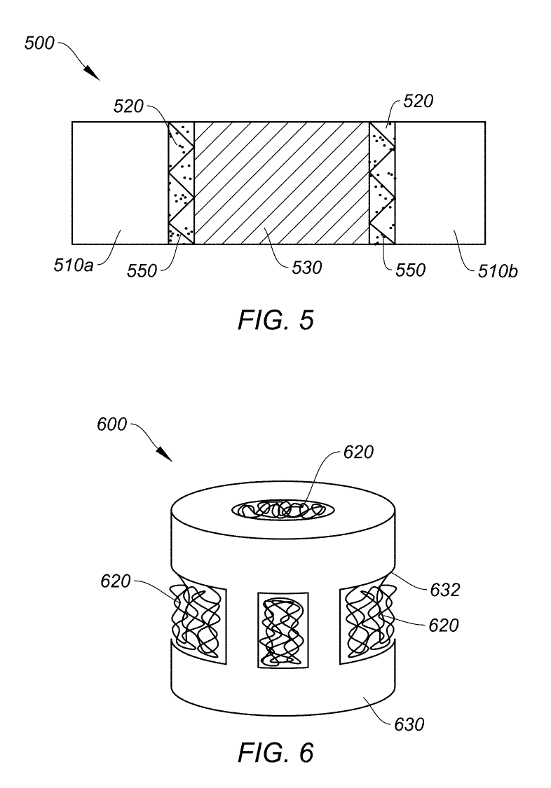

### Example 11—FIG. 5

In one exemplary embodiment, the internal cavity of the composite implantable device 500 may include flexible features to allow bending, in order to accept the graft plug or component, but may flex back to its original shape in order to keep the graft plug in place. For instance, BAG fibers may be used pre-packed with the cage component such that the fibers act as a liner or gasket and allow the BAG plug to be secured to the PEEK cage component(s) with a degree of flexibility until fully locked into place.

As illustrated, a composite implantable device 500 has a main body comprising a bioactive glass component or plug 530, similar to the one shown in FIG. 4. The ends of the plug 530 may have an interlocking junction 550 to cooperate with end caps 510a, 510b which may be formed of PEEK, for example. The interlocking junction 550 may include threads as an example. Surrounding the threads may be BAG fibers 520, as shown.

### Example 12

Rather than a PEEK cage component, an alternative may be provided in which the cage component is a collagen cage component. The collagen cage component is likewise also temperature sensitive.

### Example 13—FIG. 6

As mentioned, the cage component of the composite implantable devices may be temperature resistant or non-temperature sensitive. Such cage components may be formed of a metal, for instance. As illustrated, in another exemplary embodiment, the metallic cage 630 of the composite implantable device 600 may include open cavities 620 which may then be partially or fully filled with bone graft material 620. The bone graft material 620 may be bioactive glass in the form of fibers or morsels, as described above. If desired, allograft material may be included. The packed metallic cage and bone graft material construct 600 may be put into a collage matrix or slurry with the addition of a binder to create a multi-composition device.

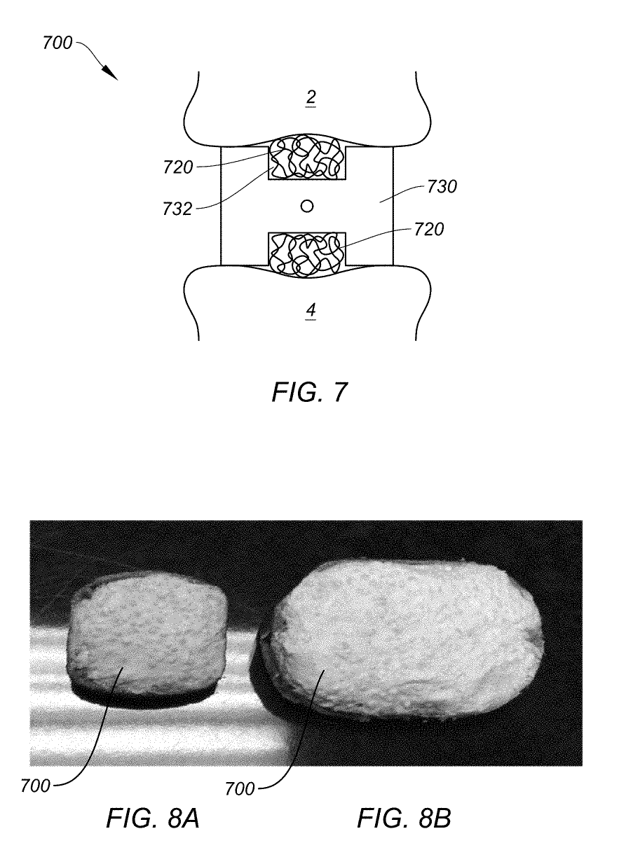

### Example 14—FIGS. 7, 8A and 8B

The device 600 of FIG. 6 described above may be modified to include overfilled caps to accommodate a particular anatomical region. For instance, in one embodiment, the metal cage component 730 of the composite implantable device 700 may be filled with a BAG material 720 such that the BAG material flows over the top of the cage to form a malleable mushroom cap. This is shown in FIG. 7 in which a metal fusion cage 730 for insertion between adjacent vertebra 2, 4 is provided. The cage 730 may have cavities or opening 732 for receiving the bone graft component, or bioactive glass fibers or morsels 720. The bone graft component 720 in this case is packed to overfill the cavities 732, thus creating this mushroom cap shape which allows the overall construct to conform to the anatomical shape of the intervertebral space. If desired, the entire device 700 can then be overwrapped and a high heat source applied (e.g., blowtorch) to shrink the device to size. This mushroom cap serves as a cushion while also allowing the device to conform to the topography of the bone surface to which the device is being attached.

Another way to form the shape is to overfill the cage component with the BAG material to form the mushroom caps at the ends inside a hyperbaric chamber. As high pressure is applied, the BAG material would shrink. The device can then be put into a mold and heat treated so that the shape is maintained. FIGS. 8A and 8B illustrate examples of devices 700 comprising overfilled bioactive glass components within a cage, then overwrapped as described above.

### Example 15

In its most basic form, the metal cage can be one that contains one or more cavities for holding the bone graft material. This material may be in an injectable form, as previously described. The material may be in a slurry, or putty, and injected using pressure to force the material into all of the cavities or empty/open spaces in the metal cage. In some instances, the slurry may be an autograft or allograft slurry. Of course, it is understood that other materials may be used, and additional agents added as previously described.

### Example 16

In another exemplary embodiment, pre-treated fibrous balls or microcapsules may be pretreated and used as filler or packing material with a metal cage. The metal cage may be an open design whereby the balls are injected or packed as already described with the fibers. In a closed design, the cage can be placed in a containment device and packed with the filler material. The packing density controls the dosage of the graft material inside the cage. If desired, once packed, the pre-treated balls may be wetted to create a temporary bond, albeit a weak one.

Alternatively, the cage may be filled with pre-wetted balls. Once filled, the construct may be dried out and the water removed by evaporation at a low temperature. This still leaves a cohesive bone graft inside the cage. Of course, the wetting agent may be something other than water, such as for example, saline, blood, bone marrow aspirate, or a similar body fluid. Still other wetting agents may be phosphate containing solutions, fluoride containing solutions, ionic containing solutions, carbonate containing solutions, soluble collagen, hyaluronic acid, and others.

### Example 17

The bone graft materials forming the bone graft component of the composite implantable device may be used with a settable agent. Suitable setting agents include, for instance, calcium silicates, calcium phosphate-based cements, calcium sulfate, pluronic/poloxmers, combinatons of PEG-based materials, calcium salts, magnesium salts, bone cements like methacrylates, polyalkylene oxides, and PEKK such as barium or strontium-based materials that bond to glass. The barium/strontium serves an additional purpose of serving as imaging markers since these materials are capable of visualization.

### Example 18

In another exemplary embodiment of a composite implantable device, a polymer cage can be built around the bone graft component. In such a scenario, the polymer cage may be softened such as by chemical softening, and then the cage built around a block of bone graft material so that the polymer cage surrounds the graft. Surface features can be built into the polymer cage to provide additional structural enhancements or interlocking features to connect multiple constructs together.

### Example 19

In still another exemplary embodiment, the opposite of Example 18 can be realized. For instance, bone graft material can surround the cage. In this scenario, the cage can be packed and then additional bone graft material placed around the cage to envelope it completely. The construct can be overwrapped to maintain its shape, as previously described. The overwrap may also increase bioactivity to improve its connectivity.

### Examples of Implantable Devices Formed of BAG

The above examples represent composite implantable devices or their components. Other implantable devices provided herein can be categorized as self-contained, or standalone, implantable devices formed of an improved bone graft material, such as for example, bioactive glass. The following are examples of these types of implantable devices.

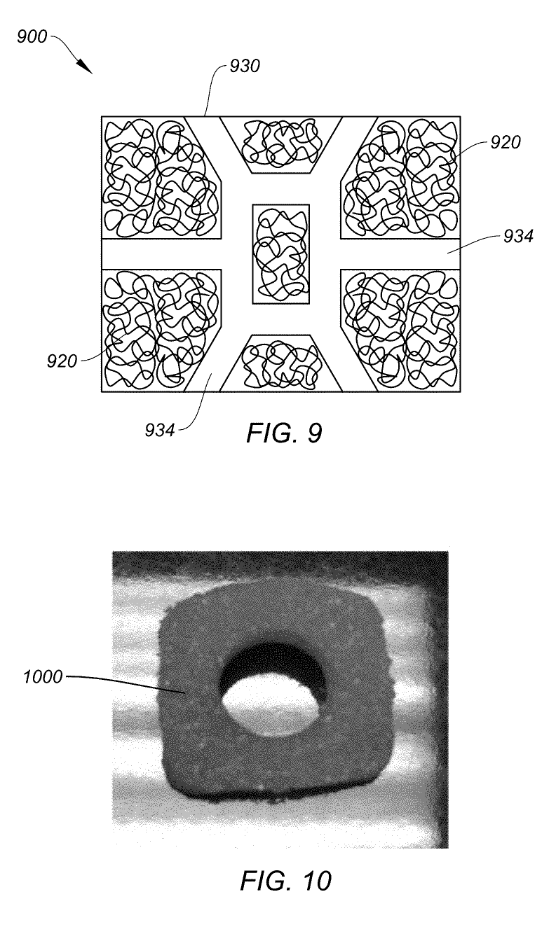

### Example 20—FIG. 9

In one exemplary embodiment, a metal truss system can be used to create a complex bone graft device 900 having interconnected voids or channels 934, which voids or channels may be open or filled. For instance, a metal truss 930 may be formed using 3D printing technology or SLM techniques. This metal truss 930 may be coated. The metal truss 930 may be filled with bone graft material 920 comprising fibers, particulates, hollow spheres in a manner as already described. Then the truss 930 can be removed to leave just the bone graft material behind. This can be accomplished by burning off the metal structure after filling, such as from sintering. What is left is a bone graft plug or molded block with complex interconnected pathways.

### Example 21—FIG. 10

In another exemplary embodiment, an implantable device 1000 formed of bioactive glass can be provided. This implantable device 1000 may comprise fibrous bioactive glass, which may or may not further include morsels, particulates or granules, and may be compacted or compressed into a particular shape or geometry. The entire device 1000 may be sintered if desired.

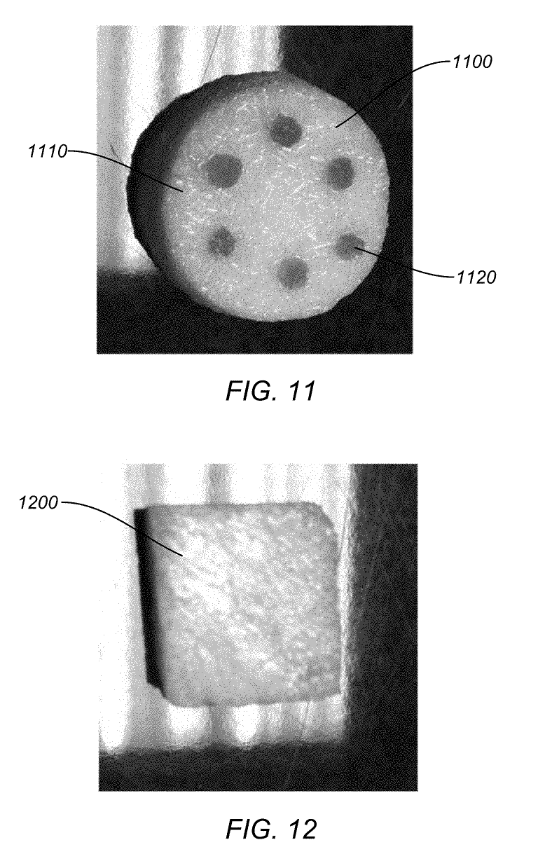

### Example 22—FIG. 11

In still another exemplary embodiment, an implantable device 1100 formed of bioactive glass can be provided. This implantable device 1100 may comprise fibrous bioactive glass 1110, which may or may not further include morsels, particulates or granules, and may be compacted or compressed into a particular shape or geometry. In addition, the fibers 1110 may be randomly oriented or aligned. Bundles 1120 of fibers 1110, which themselves may be aligned or not, may be inserted into the randomly oriented fibrous mass, as shown in FIG. 11, to form a composite bioactive glass implantable device 1110 having two different kinds of bioactive glass components, each one having a different type of fiber density, alignment and/or property. The bundles 1120 of fibers 1110 may have a different fiber density and consequently different porosity than the main body of fibrous bioactive glass 1110. The entire device 1100 may be bioresorbable, with the rate of resorption of the main body differing from the rate of resorption of the bundles. For instance, alignment of the fibers may confer directionality to the cell growth pattern. The entire device 1100 may be sintered if desired, and may also include a coating as described above. Additionally, the device may include biological agents such as growth factors as described herein.

### Example 23—FIG. 12

As previously mentioned, alignment of the fibers may lend directionality to the cell growth pattern that is desired. As such, FIG. 12 provides an implantable device 1200 comprising aligned fibers 1220 which are then optionally sintered together. These implantable devices 1200 may be load-bearing and may take any shape or geometry, size, or dimension as desired for clinical application.

Additional Characteristics

The allograft material may comprise demineralized bone matrix rather than bone chips. Furthermore, the implant may comprise one or more different glass materials to vary the composition of the implant. Additional biological agents and additives such as those previously mentioned may be utilized.

The inclusion of bioactive glass granules can be accomplished using granules having a wide range of sizes or configurations to include roughened surfaces, very large surface areas, and the like. For example, granules may be tailored to include interior lumens with perforations to permit exposure of the surface of the granule's interior. Such granules would be more quickly absorbed, allowing a tailored material characterized by differential resorbability. The perforated or porous granules could be characterized by uniform diameters or uniform perforation sizes, for example. The porosity provided by the granules may be viewed as a secondary range of porosity accorded the bone graft material or the implant formed from the bone graft material. By varying the size, transverse diameter, surface texture, and configurations of the bioactive glass fibers and granules, if included, the manufacturer has the ability to provide a bioactive glass bone graft material with selectively variable characteristics that can greatly affect the function of the material before and after it is implanted in a patient. The nano and micro sized pores provide superb fluid soak and hold capacity, which enhances the bioactivity and accordingly the repair process.

Accordingly, the engineered implant can be selectively determined by controlling compositional and manufacturing variables, such as bioactive glass fiber diameter, size, shape, and surface characteristics as well as the amount of bioactive glass granular content and structural characteristics, and the inclusion of additional additives, such as, for example tricalcium phosphate, hydroxyapatite, and the like. By selectively controlling such manufacturing variables, it is possible to provide an artificial bone graft material having selectable degrees of characteristics such as porosity, bioabsorbability, tissue and/or cell penetration, calcium bioavailability, flexibility, strength, compressibility and the like.

It is contemplated that in some embodiments, either fibers or granules, or a combination of both, may be added to the coating. The fibers or granules, which themselves may or may not be coated, would extend beyond the outer surface of the scaffold, providing a surface feature that enhances adhesion and creates a cell attachment surface.

One of the benefits of providing an ultra-porous bioactive glass material in granular form is that handling of the material can be improved. In one manner of handling the granular material, the granules may be packaged in a syringe with a carrier, and injected into the bone defect with ease. Another benefit is the additional structural effect of having a plurality of clusters closely packed together, forming additional macrostructures to the overall implant of material. Like a sieve, the openings between individual clusters can be beneficial such as when a filter is desired for various nutrients in blood or bone marrow to concentrate certain desired nutrients at the implant location.

Another implant useful for clinical applications is a kneadable, conformable, or otherwise moldable formulation or putty. Putty implants are desirable because the putty can be applied directly to the injury site by either injection or by plastering. Putty implants are also easy to handle and moldable, allowing the clinician the flexibility to form the material easily and quickly into any desired shape. In addition, the putty possesses the attributes of malleability, smearability, and injectability.

Accordingly, the bioactive glass material may be mixed with a carrier material for better clinical handling, such as to make a putty or foam implant. A pliable implant in the form of a putty may be provided by mixing the bioactive glass material with a flowable or viscous carrier. A foam implant may be provided by embedding the bioactive glass material in a porous matrix such as collagen (either human or animal derived) or porous polymer matrix. One of the advantages of a foam implant is that the porous carrier can also act as a site for attaching cells and growth factors, and may lead to a better managed healing.

The carrier material may be porous and may help contribute to healing. For example, the carrier material may have the appropriate porosity to create a capillary effect to bring in cells and/or nutrients to the implantation site, similar to the benefits that the fibers provide. The carrier material may also possess the chemistry to create osmotic or swelling pressure to bring in nutrients to the site and resorb quickly in the process. For instance, the carrier material may be a polyethylene glycol (PEG) which has a high affinity to water.

In one embodiment, the putty may have a more fluid than kneadable consistency to allow to be easily injected from a syringe or other injection system. This could be very useful in a minimally invasive system where you want as little disruption to the damaged site and to the patient as possible. For instance, a treatment may involve simply injecting the flowable putty of material into the area of bone damage using a syringe, cannula, injection needle, delivery screw, or other medical delivery portal for dispersal of injectable materials. This treatment may be surgical or non-surgical.

The combination of the ultra-porous fibrous clusters formed of bioactive glass, combined with porous bioactive glass granules and a carrier material, forms an improved putty implant over currently available putties. In one embodiment, the putty may comprise fibers and fiber clusters in a carrier material. In another embodiment, the putty may comprise fibrous clusters as previously mentioned, bioactive glass granules, and the carrier material, the fibers and granules being polymerically coated as described above. The sintered fibrous clusters as well as the bioactive glass granules may be porous, where each component may have a range or gradient of porosities throughout. The combination thus provides the putty with variable resorption rates. As mentioned above, these fiber and glass clusters may be engineered with variable porosities, allowing the customization of the putty formulation. In some embodiments, the putty includes any combination of nanopores, macropores, mesopores, and micropores.

The carrier material for the putty implant can be phospholipids, carboxylmethylcellulose (CMC), glycerin, polyethylene glycol (PEG), polylactic acid (PLA), polylactic-co-glycolic acid (PLGA), or other copolymers of the same family. Other suitable materials may include hyaluronic acid, or sodium alginate, for instance. The carrier material may be either water-based or non-water based, and may be viscous. Another carrier material alternative is saline or bone marrow aspirate, to provide a stickiness to the implant. Additives such as those described above, such as for example, silver or another antimicrobial component, may also be added to provide additional biological enhancements.

In other embodiments, the collagen may be a fully or partially water soluble form of collagen to allow the collagen to soften with the addition of fluids. In still other embodiments, the collagen may a combination of soluble and fibrous collagen. The collagen may be human derived collagen, in some instances, or animal derived collagen.

The use of sintered fiber clusters may be advantageous in some instances, because the sintering provides relative hardness to the clusters, thereby rendering the sintered clusters mechanical stronger. Their combination with the glass granules further enhances the structural integrity, mechanical strength, and durability of the implant. Because larger sized granules or clusters will tend to have longer resorption time, in previous cases the user had to sacrifice strength for speed. However, as applicants have discovered, it is possible to provide larger sized granules or clusters to achieve mechanical strength, without sacrificing the speed of resorption. To this end, ultra-porous clusters may be utilized. Rather than using solid spheres or clusters, ultra-porous clusters that have the integrity that overall larger sized clusters provide, along with the porosity that allows for speed in resorption, can be used. These ultra-porous clusters will tend to absorb more nutrients, resorb quicker, and lead to much faster healing and remodeling of the defect.

It is contemplated that the putty could be formulated for injectable delivery. For example, one manner in which to apply the putty would include a syringe containing the bioactive material that can be opened to suction into the syringe the necessary fluid to form the putty, while the same syringe can also be used to inject the as-formed putty implant. In other examples, a syringe with threaded attachments such as a removable cap may be utilized for site-specific delivery.

The use of sintered fiber clusters may be advantageous in some instances, because the sintering provides relative hardness to the clusters, thereby rendering the sintered clusters mechanical stronger. Their combination with the glass granules further enhances the structural integrity, mechanical strength, and durability of the implant. Because larger sized granules or clusters will tend to have longer resorption time, in previous cases the user had to sacrifice strength for speed. However, as applicants have discovered, it is possible to provide larger sized granules or clusters to achieve mechanical strength, without sacrificing the speed of resorption. To this end, ultra-porous clusters may be utilized. Rather than using solid spheres or clusters, ultra-porous clusters that have the integrity that overall larger sized clusters provide, along with the porosity that allows for speed in resorption, can be used. These ultra-porous clusters will tend to absorb more nutrients, resorb quicker, and lead to much faster healing and remodeling of the defect.

As previously mentioned, the fiber clusters may be sintered to provide hard clusters. Of course, it is contemplated that a combination of both sintered fiber clusters (hard granules) and unsintered clusters (soft granules) may be used in one application simultaneously. Likewise the combination of putty, foam, and clusters as described herein may be used in a single application to create an even more sophisticated porosity gradient and ultimately offer a better healing response. In some cases, solid porous clusters of the bioactive glass material may also be incorporated into the composition.

Additionally, these fibrous clusters may be encased or coated with a polymer. The coating material itself may be porous. Thus, a fibrous cluster may be further protected with a coating formed of polymer. The advantage of coating these fibrous clusters is to provide better handling since highly porous materials tend to have low strength, are prone to breakage and can become entangled. The addition of a coating having the same properties as the underlying fibrous foundation would therefore create a bead-like composition that offer yet another layer of protection as well as an additional porosity gradient.

The implant may also include surface features like granules or short wavy fibers. These added surface features can be included in the coating to provide an exterior with bioactive anchorage points to attract cellular activity and improve adhesion of the implant in situ.

In some embodiments, the fiber diameter may be in the range of about 0.1 to about 100 microns. In other embodiments, the diameter can be the range of about 0.5 to about 30 microns. In still other embodiments, the diameter can be less than about 10 microns. In one embodiment, the fiber diameter can fall within the range of between about 2 to about 10 microns.

In some embodiments, the fiber clusters may have a diameter in the range of about 0.75 to about 4.0 mm. In other embodiments, the fiber clusters may have a diameter in the range of about 2.0 to 4.0 mm.

In some embodiments, the glass granules may have a diameter in the range of about 1 to 5 mm, or about 950 microns to about 3 mm, or about 850 microns to about 3 mm. In other embodiments, the glass granules may have a diameter in the range of about 50 to 450 microns, or about 150 to 450 microns.

Although the engineered implant of the present disclosure is described for use in bone grafting, it is contemplated that the implant of the present disclosure may also be applied to soft tissue or cartilage repair as well. Accordingly, the application of the implant provided herein may include many different medical uses, and especially where new connective tissue formation is desired. One such clinical application is in the area of nucleus replacement, where the engineered implant could be inserted into the disc nucleus as part of a nucleus replacement therapy. Another suitable clinical application is for large bone defects or lesions, particularly with the addition of platelet rich plasma (PRP) to the implant composition. Even still, the implant may be applied as a bone filler such as a replacement or substitute for bone cement in bone defect repairs. A silane coating may be applied over the implant to make it more suitable in that capacity.

Other embodiments of the invention will be apparent to those skilled in the art from consideration of the specification and practice of the disclosure provided herein. It is intended that the specification and examples be considered as exemplary only, with a true scope and spirit of the disclosure being indicated by the following claims.

What is claimed is:

1. An implantable device comprising:
a main body comprising a plurality of compressed bioactive glass fibers and at least one bundle of compressed bioactive glass fibers within the main body, the main body and the at least one bundle having different fiber densities and porosities;
wherein the device has a shape and geometry configured for insertion between adjacent bone segments to facilitate bone fusion.

2. The implantable device of claim 1, further comprising a plurality of bioactive glass particulates in the main body or the bundle.

3. The implantable device of claim 1, wherein the bioactive glass fibers of the main body or at least one bundle are randomly oriented.

4. The implantable device of claim 1, wherein the bioactive glass fibers of the main body or at least one bundle are aligned with respect to one another.

5. The implantable device of claim 1, wherein the bioactive glass fibers of the main body or at least one bundle are sintered together.

6. The implantable device of claim 1, wherein the device comprises a plurality of bundles of compressed bioactive glass fibers within the main body.

7. The implantable device of claim 6, wherein the plurality of bundles of compressed bioactive glass fibers are equidistantly spaced apart from one another within the main body.

8. The implantable device of claim 1, wherein the adjacent bone segments are vertebral bodies.

9. The implantable device of claim 1, wherein the device is porous.

10. The implantable device of claim 1, wherein the device is bioresorbable.

11. The implantable device of claim 10, wherein the rate of resorption is different for the main body and the at least one bundle.

12. The implantable device of claim 1, wherein the device is configured to be load-bearing.

13. The implantable device of claim 1, wherein the device is shaped as a cylinder.

14. The implantable device of claim 1, further including a coating over the main body.

15. The implantable device of claim 14, wherein the coating is heat wrapped over the main body.

16. The implantable device of claim 1, further including a biological agent.

17. The implantable device of claim 16, wherein the biological agent may be selected from the group consisting of growth factors, synthetic factors, recombinant factors, allogenic factors, stem cells, demineralized bone matrix (DBM), or cell signaling agents.
